(19) United States
(12) Patent Application Publication
(10) Pub. No.: US 2017/0246636 A1
(43) Pub. Date: Aug. 31, 2017

(54) POINT-OF-CARE BIOMARKER ASSAY APPARATUS ARRANGED FOR MEASURING A PRESENCE OR CONCENTRATION OF A BIOMARKER IN A SAMPLE

(71) Applicant: CitiusBio B.V., Nijmegen (NL)

(72) Inventor: Christianus Franciscus Rosmalen, Nijmegen (NL)

(73) Assignee: CitiusBio B.V., Nijmegen (NL)

(21) Appl. No.: 15/510,318

(22) PCT Filed: Sep. 10, 2015
(86) PCT No.: PCT/NL2015/050628

§ 371 Date: Mar. 10, 2017

(30) Foreign Application Priority Data
Sep. 10, 2014 (NL) 2013450

(51) Int. Cl. B01L 7/00; B01L 9/00; G01N 35/02; C12N 15/10; G01N 35/04; G01N 33/49; C12Q 1/68

(52) U.S. Cl. CPC B01L 7/52; G01N 33/49; B01L 9/527; C12Q 1/686; C12Q 1/6806; C12N 15/1013; G01N 35/04; B01L 9/543; G01N 35/026; G01N 2035/00425

(57) ABSTRACT

Point-of-care biomarker assay apparatus arranged for measuring a presence or concentration of a biomarker in a sample, said biomarker assay apparatus comprising cartridge receiving means arranged for receiving at least one cartridge having multiple chambers designed for receiving a plurality of liquid media comprising said sample, labelled binding reagent, magnetic beads reagent and wash buffer, a sample distribution unit arranged for processing pipetting steps with said chambers, thereby providing a liquid reactant mixture in one or more of said chambers, a magnetic coil assembly arranged for applying a magnetic field to said liquid reactant mixture for separating biomarkers bound to said magnetic beads and said labelled binding reagent, from said reactant mixture, a photo detector or assembly arranged for measuring said presence or concentration of said labelled binding reagent, a control unit arranged for controlling said processing pipetting steps with said chambers, and for controlling said sample distribution unit along said chambers according to a test protocol, wherein said test protocol comprises an order of subsequent processing steps performed in said plurality of chambers to be processed by said sample distribution unit, said processing steps comprising one or more processing pipetting steps by the sample distribution unit and one or more processing incubation steps of the reaction mixture within any of the chambers wherein the control unit being programmed for performing multiple, distinct test protocols.

The present invention relates to a point-of-care biomarker assay apparatus arranged for measuring a presence or concentration of a biomarker in a sample.

In recent years, a method for separating a target biomarker present in a sample is developed in the field of biotechnology by using magnetic beads. This method is known for a wide range of applications such as immunoassay, DNA hybridization, Polymerase Chain Reaction (PCR), separation of cells, separation or washing of protein, etc.

Separation of a biomarker by using magnetic beads has been achieved by using the following pipetting steps. A piston in a syringe-like cylinder is lifted to withdraw a liquid containing magnetic beads into the cylinder. During the withdrawal process, the magnetic beads in the liquid are attracted to magnets arranged on the outer side of the cylinder or of a container of the distribution tip portion. Subsequently, the piston is lowered to discharge the liquid, leaving the magnetic beads adhering to the inner surface of the distribution tip portion. In this manner, the magnetic beads are separated as the magnetic beads are capable of binding to the biomarker, the biomarker can be separated through the beads.

Then, with the magnetic particles separated from the liquid, the piston is raised again to withdraw another liquid into the tip thereby washing the magnetic beads. The magnets are then either turned of or physically removed, such that the magnetic beads are suspended in the liquid. In this condition, the piston is lowered to discharge the magnetic beads-suspended liquid from the tip.

Before the separation of the magnetic particles, an immunoreaction may take place between the biomarker present in the sample and a (chemi)luminescent or fluorescent labelled antibody. The presence or concentration of the biomarker present in the sample may then, ultimately, be determined using a photomultiplier by measuring the emitted light of the (chemi)luminescent or fluorescent reaction.

Known biomarker assay apparatuses are arranged to detect the presence or concentration of the biomarker in a laboratory environment. Hence, a sample tube, with for example plasma, is placed entirely in the apparatus. The reactions will be performed with reagents that are present in a separate cartridge that is also placed in the apparatus, containing sufficient reagents to perform multiple measurements. Usually, it may take several hours before a result is obtained, i.e. before the amount or concentration present in the sample is determined by the biomarker assay apparatus.

These known types of apparatuses are not suitable to be used as a point-of-care apparatus. Point-of-care testing, or bed-side testing, is defined as medical testing at or near the site of patient care. This includes medical blood tests, which can be performed at the bedside.

The drawback of the known biomarker assay apparatuses is that they are too cumbersome, i.e. not portable to a patients bed. Further, a key feature of point-of-care testing is that the results of the test may be obtained within just a couple of minutes such that appropriate action e.g. treatment by a physician can be executed immediately.

Known point-of-care apparatuses already exist in the form of hand-held devices. These devices, however, are based on another method for determining the presence and/or the concentration of the biomarker in the blood, which is in fact relatively inaccurate.

It is therefore an objective of the present invention to provide a point-of-care biomarker assay apparatus that is suitable to be used as an apparatus to be carried to the bedside of the patient and to perform tests, for example to detect presence or concentration of an biomarker in a blood sample, within just a couple of minutes.

This objective is achieved by providing a point-of-care biomarker assay apparatus arranged for measuring a presence or concentration of a biomarker in a sample, said biomarker assay apparatus comprising:

cartridge receiving means arranged for receiving at least one cartridge having multiple chambers designed for receiving a plurality of liquid media comprising:

the sample comprising said biomarker,

labelled binding reagent, capable of binding to said biomarker, or capable of competing with said biomarker,

magnetic beads reagent, capable of binding to said biomarker and/or capable of binding competing binding reagent,

wash buffer, and

at least one substrate capable of initiating a light emission from the labelled binding reagent;

a sample distribution unit arranged for processing pipetting steps with said chambers, thereby providing a liquid reactant mixture in one or more of said chambers wherein the biomarker is allowed to bind to the labelled binding reagent and the magnetic beads reagent in a processing incubation step or to compete with the labelled binding reagent and bind the magnetic bead reagent in a processing incubation step;

wherein said sample distribution unit further comprises at least one precision pipet for performing said pipetting steps, each at least one precision pipet comprising

a hollow elongated tube arranged to detachably couple to a pipet tip provided at said at least one cartridge;

an adjustable volume container, coupled to said hollow elongated tube, wherein said sample distribution unit is arranged to set a volume of said adjustable volume container for pipetting a set volume of liquid media from said chambers;

wherein said sample distribution unit is arranged to dispose said pipet tip at said at least one cartridge after said pipetting steps have been performed;

a magnetic coil assembly arranged for applying a magnetic field to said liquid reactant mixture for separating biomarkers bound to said magnetic beads reagent and/or said labelled binding reagent, from said reactant mixture;

a photo multiplier tube arranged for measuring said presence or concentration of labelled binding reagent bound to the biomarker, or bound to the magnetic beads in case of a competitive assay format;

a control unit arranged for controlling said processing pipetting steps with said chambers, and for controlling said sample distribution unit along said chambers according to a test protocol, wherein said test protocol comprises:

an order of subsequent processing steps performed in said plurality of chambers to be processed by said sample distribution unit, said processing steps comprising one or more processing pipetting steps by the sample distribution unit and one or more processing incubation steps of the reaction mixture within any of the chambers;

wherein said control unit being programmed for performing multiple, distinct test protocols,

heating means arranged for heating each of said chambers of said at least one cartridge, simultaneously and uniformly, when placed in the apparatus, to a predefined temperature.

In accordance with the present invention, many different types of biomarkers may be used, suitable to be used with the point-of-care biomarker assay apparatus according to the present invention, like Ribonucleic acid (RNA), Micro Ribonucleic acid (Mirco-RNA), Deoxyribonucleic acid (DNA), metabolites, peptides, proteins, etc.

As such the labelled binding agent may be an antibody, also known as an immunoglobulin, which is a large Y-shape protein produced by plasma cells that is used to identify and neutralize foreign objects such as for example bacteria and viruses. The antibody recognizes a unique part of the foreign target/biomarker, called an antigen, in the sample.

The labelled binding agent may also be an antigen.

The labelled binding agent may also be DNA-binding proteins (or RNA-binding proteins), which are proteins composed of DNA-binding domains, and thus have a specific or general affinity for either single or double stranded DNA.

In accordance with the present application, an assay apparatus is an investigative (analytical) procedure for qualitatively assessing or quantitatively measuring the presence, amount or concentration of a biomarker in a sample. At present, the assay involves the addition of a fixed amount of exogenous reactants (or in excess), such as labelled binding reagent, for example enzymes. By detecting or measuring the labelled binding reagent in the assay outcome, the unknown quantity of the biomarker may be derived.

To do so, the assay apparatus is arranged to perform multiple pipetting steps during the biochemical reaction process, wherein the sample is subjected to distinct chemical reaction steps each time. For example, the sample is dispensed into a chamber having magnetic beads reagent, or the other way around, resulting in a reactant mixture wherein the magnetic beads reagent have bound to the biomarkers. Further, labelled binding reagents are provided to the liquid reactant mixture and the binding reagent may also bind to the biomarkers in the sample.

In order to separate the biomarkers having attached to the magnetic beads reagent and/or the labelled binding reagent from the liquid reactant mixture, a magnetic coil assembly is actuated to apply a magnetic field to the liquid reactant mixture, which magnetic field attracts the magnetic beads reagent.

After this, the excess of unbound labelled binding reagent is washed away by means of emptying the pipet tip (except the magnetic beads reagents) and/or with diluting the remaining volume of unbound labelled binding reagents by means of adding and removing a wash solution.

After this, the amount of labelled binding reagent is made measureable by adding one or two substrates (chemiluminescence) or applying a light source (fluorescence)

Next, a photo detector assembly is utilized for measuring the presence or concentration of the labelled binding reagent. As these labelled binding reagents are each bound to a biomarker, or have competed with the biomarker, the amount of light measured is proportional or inversely proportional with the concentration of the biomarkers.

It was the insight of the inventors that the use of any point-of-care biomarker assay apparatus is made more advantageous in case the point-of-care biomarker assay apparatus is capable of performing multiple, distinct test protocols. As such, the operator is able to test the samples for the presence of different biomarkers, even if this would require a different test protocol.

According to the present invention, a test protocol comprises the subsequent order of processing steps to be performed in the plurality of chambers to be processed by the sample distribution unit, wherein the processing steps comprise one or more pipetting steps by the sample distribution unit and one or more incubation steps of the reaction mixture within any of the chambers.

By using different test protocols, each biomarker assay can be optimized within it's own limits, which enables to design biomarker assays with an optimal balance between shortening the time to result and high sensitivity.

Different test protocols are, for example, used for immunoassay, i.e. a test that measures the presence or concentration of a biomarker in a sample through the use of an antibody as explained above, and for polymerase chain reaction, PCR, i.e. a technology in molecular biology used to amplify a single or a few copies of a piece of DNA across several orders of magnitude, generating a multitude of copies of a particular DNA sequence. As such, the point-of-care biomarker assay apparatus according to the present invention is able to process test protocols for immunoassay techniques as well as for PCR techniques.

As such, the labelled binding agent may comprise any of labelled antibody, labelled DNA and labelled RNA.

Further, the point-of-care assay apparatus comprises heating means for heating each of said chambers of said at least one cartridge, simultaneously and uniformly, when placed in the apparatus, to a predefined temperature, in order to obtain the results of the test within just a couple of minutes. It was the insight of the inventor that all liquid media should be heated at the same time as in such a case—1) the predefined, relatively high, temperature of each of the liquid media ensures that the time required for any of the chemical reactions is reduced—2) it does not matter whether a cartridge is retrieved from a refrigerator or from a facility at room temperature—3) the heated temperature enables that the process can be repeated anywhere in the world, under the same conditions.

The above thus enables that appropriate action e.g. treatment by a physician can be executed immediately.

In accordance with the present invention, the sample distribution unit comprises at least one precision pipet for performing the pipetting steps, wherein each of the at least one precision pipet comprises a hollow elongated tube arranged to detachably couple to a pipet tip provided at said at least one cartridge, and an adjustable volume container, coupled to said hollow elongated tube, wherein said sample distribution unit is arranged to set a volume of said adjustable volume container for pipetting a set volume of liquid media from said chambers.

The above increases the usability of the apparatus, i.e. it makes the apparatus suitable for different type of tests, and for different amounts of liquid media in the chambers.

As mentioned above, the goal of the present invention is to provide for an point-of-care biomarker assay apparatus capable of quickly, and accurately, perform tests. The accuracy of the apparatus is ensured by using a photomultiplier tube assembly arranged for measuring the presence or concentration of labelled binding reagent bound to the biomarker, or bound to the magnetic beads in case of a competitive assay format. The inventors found that photomultiplier tube assemblies comprise extremely sensitive detectors of light in the ultraviolet, visible and/or near-infrared ranges of the electromagnetic spectrum. These detectors multiply the current produced by incident light by as much as millions of times, enabling, for example, individual photons to be detected when the incident flux of light is very low.

According to the present invention, the point-of-care biomarker assay apparatus is also suitable for measuring a presence or concentration of a biomarker in a sample based on a competitive assay approach. Several different types of competitive assay approaches exist. In a competitive, homogeneous immunoassay, the unlabelled biomarker in the sample competes with labelled biomarkers, i.e. antigens, to bind to the magnetic beads. The amount of labelled, unbound biomakers may then be measured. In theory, the more unlabelled biomarker in the sample, the more labelled biomarker gets competed off and hence the amount of labelled, unbound biomarker is proportional to the amount of biomaker in the sample. As in a competitive, homogeneous assay, in a competitive, heterogeneous immunoassay unlabeled biomaker in a sample competes with labelled biomarker to bind an antibody present on the magnetic beads. In the heterogeneous assays the labelled, unbound biomarker is separated or washed away, and the remaining labelled, bound biomarker is measured.

In an example, the heating means comprise a plurality of metal heating elements arranged to be in contact with said at least one cartridge, when received in said cartridge receiving means, wherein said heating means are arranged to heat a received cartridge to said predefined temperature by heating said metal heating elements.

Here, the predefined temperature is preferably between 28-40 degrees Celsius, preferably between 34-38 degrees Celsius, even more preferably approximately 35 degrees Celsius.

The predefined temperature may also be between 30-95 degrees Celsius, between 45-65 degrees Celsius, and around 72 degrees Celsius.

In a preferred example, the adjustable volume container comprises an adjustable volume between 5 μl-1000 μl.

In another example, the sample distribution unit is arranged to set a volume of said adjustable volume container, for each pipetting step separately, for pipetting a set volume of liquid media from said chambers.

In yet a further example, the assay apparatus comprises said at least one cartridge, and wherein said pipet tips provided at said at least one cartridge are further arranged to provide level detection for detecting an amount of liquid media in said chambers and to provide clot detection for detecting clots in said liquid media.

Here, the assay apparatus may be arranged to provide an alert signal in case a detected amount of liquid media in any of said chambers is below a predetermined threshold.

The assay apparatus may also be arranged to provide a cloth alert signal in case detected clots in said liquid media in any of said chambers are above a predetermined threshold.

In an example, the processing steps comprise at least one washing step, wherein the control unit may be programmed for setting a duration of the at least one washing step.

In an example, the cartridge receiving means are arranged for receiving a single cartridge.

The inventors noted that a biomarker assay apparatus having cartridge receiving means arranged for receiving a single cartridge is beneficial for point-of-care type of testing, as for point-of-care, usually, only one test at a time needs to be performed such that it may be superfluous to provide an apparatus capable of receiving multiple cartridges at a same time.

A known biomarker assay apparatus is equipped with cartridge receiving means arranged for receiving multiple cartridges at the same time. This, however, may not be beneficial for point-of-care testing, as it is often superfluous to perform the same test at multiple cartridges in parallel. As such, the known biomarker assay apparatus is not as versatile deployable as the point-of-care biomarker assay apparatus according to the present invention.

A further advantage of having cartridge receiving means arranged for receiving only a single cartridge is that the physical dimension of the apparatus are reduced such that it is made more convenient to transport the apparatus to and from a patients bedside and to take less space in in general overcrowded places such as an emergency room.

A yet further advantage of having cartridge receiving means arranged for receiving only a single cartridge is that the risk of mixing up samples of patients is reduced. As only one cartridge is provided at a time, the results obtained for the sample contained in that cartridge is associated with just one patient, thereby removing any possible errors in the mix up of samples. As such, any mix up in the samples is prevented.

In a further example, there is provided a point-of-care biomarker assay apparatus arranged for measuring a presence or concentration of a biomarker in a sample, the biomarker assay apparatus comprising:

cartridge receiving means arranged for receiving at least one cartridge having multiple chambers designed for receiving the plurality of liquid media comprising the sample, labelled binding reagent, magnetic beads reagent, substrate and wash buffer;

a sample distribution unit arranged for processing pipetting steps with the chambers, thereby providing a reactant mixture in one or more of the chambers;

a magnetic coil assembly arranged for applying a magnetic field to the liquid reactant mixture for separating biomarkers bound to the magnetic beads reagent and the labelled binding reagent, from the liquid reactant mixture;

a photo detector assembly arranged for measuring the presence or concentration of the labelled binding reagent;

a control unit arranged for controlling the processing pipetting steps with the chambers, and for controlling the sample distribution unit along the chambers according to a test protocol, wherein the test protocol comprises:

an order of subsequent processing steps performed in the plurality of chambers to be processed by the sample distribution unit, the processing steps comprising one or more processing pipetting steps by the sample distribution unit and one or more processing incubation steps of the reaction mixture within any of the chambers, wherein the apparatus further comprises:

heating means arranged for heating the chambers of the at least one cartridge, when placed in the apparatus, to a predefined temperature.

During the above mentioned different reaction steps, it may be necessary to heat the reactant mixture to a predefined temperature such that the chemical reaction is performed effectively. In order to reduce the total time required to obtain the test results, the inventors noted that it is more efficient to heat the chambers containing liquids to a predefined temperature instead of heating the liquid reactant mixture to the predefined temperature. As such, during the pipetting steps, it is not necessary to heat the magnetic beads reagent, the labelled binding reagent, at least one substrate and the wash buffer as these have already been heated to the predefined temperature, by heating the chambers of the at least one cartridge to the predefined temperature.

The advantage hereof is that the total time for obtaining the measurement results, i.e. the presence or concentration of biomarker in a sample, is reduced, making the apparatus more suitable to be used for point-of-care.

In an example, the heating means are arranged for heating the chambers by heating the at least one cartridge to the predefined temperature.

It might be advantageous, i.e. pragmatically wise, to heat the complete at least one cartridge instead of heating each of the chambers separately.

In another example, the predefined temperature is between 28-40 degrees Celsius, preferably between 34-38 degrees Celsius, even more preferably approximately 35 degrees Celsius.

The heating means may further be arranged to heat the chambers to a predefined temperature between 85-100 degrees Celsius, preferably between 90-100 degrees Celsius, even more preferably around 95 degrees Celsius.

The inventors noted that the set point for the predefined temperature should be different, depending on the technique used, for example for an immunoassay technique or a PCR technique. Chemical reactions occurring during an immunoassay process are usually performed around 35 degrees Celsius, while chemical reactions occurring during a PCR process are performed up to around 95 degrees Celsius. In order to cope with both techniques, it might be advantageous in case the heating means are arranged to either heat the chambers to a predefined temperature of 35 degrees Celsius or to heat the chambers, or the complete at least one cartridge, to a predefined temperature of 95 degrees Celsius.

A PCR process comprises different steps, each of which may be performed at a different temperature range. A first step is, for example, a denaturation process which causes DNA melting of the DNA template by disrupting the hydrogen bonds between complementary bases. A denaturation process is usually performed at about 94 degrees Celsius. A second step is, for example, an annealing process which allows for annealing of the primers to the single-stranded DNA template. An annealing process is typically performed at a temperature between 45-65 degrees Celsius. A third step is, for example, an elongation step which is typically performed at around 72 degrees Celsius.

As such, the heating means may even further be arranged to heat heat the chambers to a predefined temperature between 85-100 degrees Celsius, and a predefined temperature between 45-65 degrees Celsius and at a temperature around 72 degrees Celsius for a PCR process, as well as to a predefined temperature of around 35 degrees Celsius for an immunoassay process.

In another example, the cartridge receiving means are arranged for receiving at least one cartridge having between 5-20 chambers, more preferably between 10-15 chambers, and even more preferably around 12 chambers.

In a further example, there is provided a point-of-care biomarker assay apparatus arranged for measuring a presence or concentration of a biomarker in a sample, the biomarker assay apparatus comprising:

cartridge receiving means arranged for receiving at least one cartridge having multiple chambers designed for receiving a plurality of liquid media comprising the sample, labelled binding reagent, magnetic beads reagent and wash buffer;

a sample distribution unit arranged for processing pipetting steps with the chambers, thereby providing a liquid reactant mixture in one or more of the chambers;

a magnetic coil assembly arranged for applying a magnetic field to the liquid reactant mixture for separating biomarkers bound to the magnetic beads and the labelled binding reagent, from the reactant mixture;

a photo detector assembly arranged for measuring the presence or concentration of the labelled binding reagent;

a control unit arranged for controlling the processing pipetting steps with the chambers, and for controlling the sample distribution unit along the chambers according to a test protocol, wherein the test protocol comprises:

an order of subsequent processing steps performed in the plurality of chambers to be processed by the sample distribution unit, the processing steps comprising one or more processing pipetting steps by the sample distribution unit and one or more processing incubation steps of the reaction mixture within any of the chambers;

wherein the sample distribution unit comprises a hollow elongated tube through which the sample distribution unit performs the applying of the pressure, wherein the hollow elongated tube is arranged to couple to at least one pipet tip provided at the at least one cartridge.

The advantage of the above provided example is that the apparatus does not need to be provided with pipet tips, as a pipet tip is already provided on the at least one cartridge. Normally, each time a test has been performed, i.e. the amount of or the concentration of the biomarker present in the sample has been measured, pipet tips are renewed such that the test results of a subsequent test is not affected by the residual volume in the pipet tips used for the test. The inventors noted that it is more convenient if the at least one cartridge is provided with one or more pipet tips such that the residual volume is removed simultaneously with the removal of the at least one cartridge. It is therefore not necessary to fill the apparatus with spare pipet tips or the like.

In a further example, the point-of-care biomarker assay apparatus comprises:

wireless sensor means arranged for wirelessly reading out a tag provided on the at least one cartridge.

Here, the control unit may be arranged for automatically setting a specific test protocol based on the read out tag.

The wireless sensor means may be arranged to read out, for example, a Radio Frequency IDentification, RFID, tag provided on the at least one cartridge.

In a further example, the washing duration is approximately 20-60 seconds, more preferably between 25-40 seconds, even more preferably around 30 seconds.

The point-of-care biomarker assay apparatus, for example the control unit, may comprise a processor and a memory, wherein the memory comprises instructions executable by the processor, whereby the control unit is operative to perform any of the control steps directed to the control unit, as disclosed with the examples provided above.

In the context of the present invention, a module, device, equipment, or the like may also be implemented as a computer program running on the processor.

The expressions, i.e. the wording, of the different aspects comprised by the point-of-care biomarker assay apparatus according to the present invention should not be taken literally. The wording of the aspects is merely chosen to accurately express the rationale behind the actual function of the aspects.

In accordance with the present invention, different aspects applicable to the above mentioned examples of the apparatus, including the advantages thereof, correspond to the aspects which are application to the method as explained below and in according with the present invention.

In a second aspect of the invention, there is provided a method for measuring a presence or concentration of a biomarker in a sample using a point-of-care biomarker assay apparatus according to any of the claims, the method comprising:

receiving, by the cartridge receiving means, the at least one cartridge having multiple chambers designed for receiving a plurality of liquid media comprising the sample, labelled binding reagent, magnetic beads reagent and wash buffer;

processing, by the sample distribution unit, pipetting steps with the chambers, thereby providing a liquid reactant mixture in one or more of the chambers;

applying, by the magnetic coil assembly, a magnetic field to the liquid reactant mixture for separating biomarkers bound to the magnetic beads and the labelled binding reagent, from the reactant mixture;

measuring, by the photo detector assembly, the presence or concentration of the labelled binding reagent;

controlling, by the control unit, the processing pipetting steps with the chambers, and for controlling the sample distribution unit along the chambers according to a test protocol, wherein the test protocol comprises:

an order of subsequent processing steps performed in the plurality of chambers to be processed by the sample distribution unit, the processing steps comprising one or more processing pipetting steps by the sample distribution unit and one or more processing incubation steps of the reaction mixture within any of the chambers;

characterized in that

the control unit being programmed for performing multiple, distinct test protocols.

The method may be used with any type of labelled binding agent like labelled antibody, labelled antigen, labelled DNA and labelled RNA.

The processing steps may comprise at least one washing step, wherein the control unit may be programmed for setting a duration of the at least one washing step.

In an example thereof, the method further comprises the step of:

heating, by heating means comprised by the apparatus, the chambers of the at least one cartridge, when placed in the apparatus, to a predefined temperature.

The step of heating may further comprise:

heating, by heating means comprised by the apparatus, the chambers by heating the at least one cartridge to the predefined temperature.

The predefined temperature may lay between 28-40 degrees Celsius, preferably between 34-38 degrees Celsius, even more preferably approximately 35 degrees Celsius, between 85-110 degrees Celsius, preferably between 90-100 degrees Celsius, even more preferably around 95 degrees Celsius, or combinations thereof.

In another example, the method further comprises the step of:

wirelessly reading out, by wireless sensor means provided in the apparatus, a tag provided on the at least one cartridge.

Here, the control unit may automatically set a specific test protocol based on the read out tag.

The above-mentioned and other features and advantages of the invention will be best understood from the following description referring to the attached drawings. In the drawings, like reference numerals denote identical parts or parts performing an identical or comparable function or operation.

The invention is not limited to the particular examples disclosed below in connection with a particular type of immunoassay or PCR.

### BRIEF DESCRIPTION OF THE DRAWINGS

FIG. 1 is a block diagram illustrating an embodiment of a point-of-care biomarker assay apparatus according to the present invention.

FIG. 2 is a flowchart illustrating an embodiment of the method steps according to the present invention.

FIG. 3 is an example of a cartridge to be used with the point-of-care biomarker assay apparatus according to the present invention.

### DETAILED DESCRIPTION

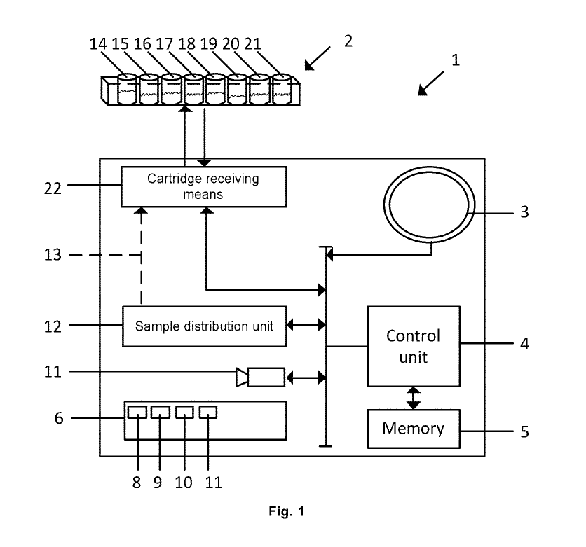

FIG. 1 is a block diagram illustrating an embodiment of a point-of-case biomarker assay apparatus 1 according to the present invention.

The point-of-care biomarker assay apparatus is arranged, or designed, for measuring a presence or concentration of a biomarker in a sample. According to the present invention, different types of biomarkers may be detected like Ribonucleic acid (RNA), Micro Ribonucleic acid (Mirco-RNA), Deoxyribonucleic acid (DNA), metabolites, peptides, proteins, etc.

The point-of-care biomarker assay apparatus comprises cartridge receiving means 22 arranged for receiving at least one cartridge 2 having multiple chambers 14-21 designed for receiving a plurality of liquid media.

The first chamber 14 may, for example contain the sample comprising the biomarker, the second chamber 15 may, for example, contain the labelled binding reagent, capable of binding to the biomarker, or capable of competing with the biomarker, the third chamber 16 may, for example, contain magnetic beads reagent, capable of binding to the biomarker and/or capable of binding competing binding reagent, the fourth chamber 17 may, for example, contain a wash buffer for washing any substance, a fifth chamber 18 may, for example, contain at least one substrate capable of initiating a light emission from the labelled binding reagent. The remaining chambers 19-21 may, for example, not be used, or may contain other reagents to be used during an immunoassay test protocol.

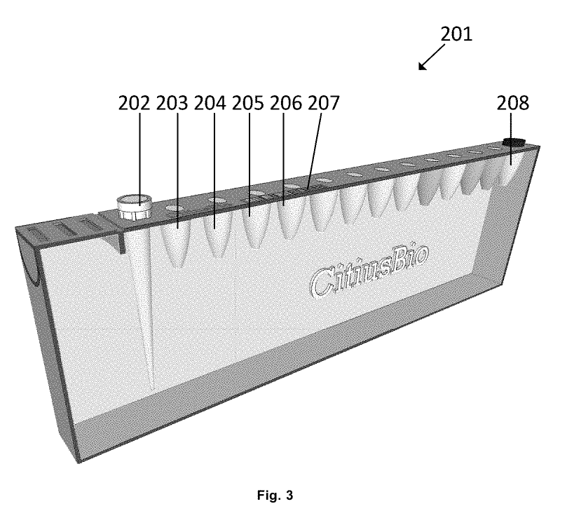

In accordance with the present invention, the at least one cartridge 2 may comprise a pipet tip (see FIG. 3) which is to be coupled to the sample distribution unit 12. After the test protocol is completed, the pipet tip may be returned in the at least one cartridge 2, and the at least one cartridge 2 including the pipet tip may be disposed of.

The point-of-care biomarker assay apparatus 1 further comprises the sample distribution unit 12 which is arranged for processing pipetting steps with the chambers 14-21, thereby providing a liquid reactant mixture in one or more of the chambers 14-21 wherein the biomarker is allowed to bind to the labelled binding reagent and the magnetic beads reagent in a processing incubation step or to compete with the labelled binding reagent and bind the magnetic bead reagent in a processing incubation step.

The sample distribution unit 12 may be directly, mechanically, coupled, or via electronic means, 13 such that the sample distribution unit 12 is able to directly control the cartridge receiving means 22.

A magnetic coil assembly 3 is further present in the point-of-care biomarker assay apparatus, which coil assembly 3 is arranged for applying a magnetic field to the liquid reactant mixture for separating biomarkers bound to the magnetic beads reagent and/or the labelled binding reagent, from the reactant mixture. This process may, preferably, take place in the pipet tips comprised on each of the chambers 14-21. Alternatively, this process may take place in each of the chambers 14-21 or each of the hollow elongated tubes present on the sample distribution unit 12.

Finally, a photo detector assembly 11 is comprised in the point-of-care biomarker assay apparatus, which photo detector assembly 11 is arranged for measuring the presence or concentration of labelled binding reagent bound to the biomarker, or bound to the magnetic beads in case of a competitive assay format.

All the above mentioned components of the point-of-care biomarker assay apparatus are under control of a control unit 4, coupled to a memory 5, which control unit 4 is arranged for controlling processing pipetting steps with the chambers 14-21, and for controlling the sample distribution unit 12 along the chambers 14-21 according to one of a multitude of test protocols 8-11 comprised in the test protocol setting block 6. The present figure only displays as an example four test protocols. In practice many more different test protocols may be available to be used with the point-of-care biomarker assay apparatus.

A test protocol 8-11 comprises an order of subsequent processing steps to be performed in the plurality of chambers to be processed by the sample distribution unit, wherein the processing steps comprising one or more processing pipetting steps by the sample distribution unit and one or more processing incubation steps of the reaction mixture within any of the chambers.

In accordance with the present invention, the control unit 4 is programmed for performing multiple, distinct test protocols. As such, the control unit may alter, amend, select and/or determine a test protocol 8-11 to be used for a particular immunoassay process.

In an example, the point-of-care biomarker assay apparatus comprises wireless sensor means in the form of Radio Frequency Identification means, which are arranged for wirelessly reading out a tag provided on the at least one cartridge 2. Based on the tag read out, the control unit may automatically select, amend, or create a specific test protocol 8-11 to be used during the immunoassay procedure.

One of the advantages of the present invention is that the point-of-care biomarker assay apparatus is suitable to be used for several, distinct and different types of assay procedures, which require different test protocols.

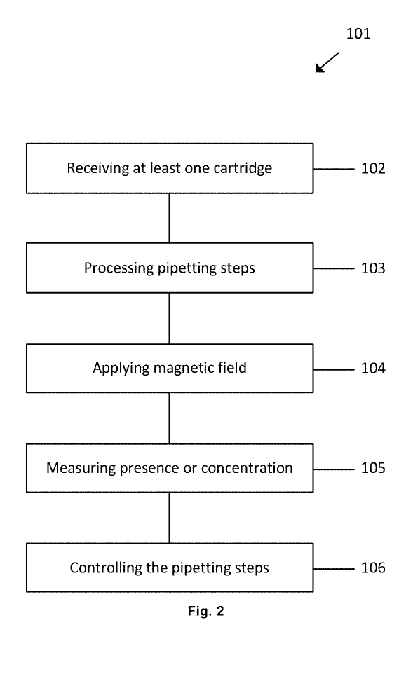

FIG. 2 is a flowchart illustrating an embodiment of the method steps 101 according to the present invention.

The method 101 is utilized for measuring a presence or concentration of a biomarker in a sample using a point-of-care biomarker assay apparatus according to any of the embodiments described above.

The method comprises several distinct method steps, such as receiving 102, by the cartridge receiving means 22, at least one cartridge 2 having multiple chambers 14-21 designed for receiving a plurality of liquid media.

Different types of liquid media may be contained in the chambers, such as, but not limited to, the sample comprising the biomarker, labelled binding reagent, capable of binding to the biomarker, magnetic beads reagent, capable of binding to the biomarker and, optionally wash buffer.

A further method step comprises processing 103, by the sample distribution unit 12, pipetting steps with the chambers 14-21, thereby providing a liquid reactant mixture in one or more of the chambers 14-21 wherein the biomarker is allowed to bind to the labelled binding reagent and the magnetic beads reagent in a processing incubation step.

Next, a magnetic field is applied 104, by the magnetic coil assembly 3, to the liquid reactant mixture for separating biomarkers bound to magnetic beads and the labelled binding reagent, from the reactant mixture.

Finally, the presence or concentration of the labelled binding reagent bound to the biomarker is measured 105, by the photo detector assembly 11.

According to the present invention, an inventive merit is obtained by the step of controlling 106, by the control unit 4, the processing pipetting steps with the chambers 14-21, and by the step of controlling the sample distribution unit along the chambers 14-21 according to a test protocol 8-11.

The test protocol 8-11 is tuned to a specific assay process, such that a test protocol 8-11 comprises an order of subsequent processing steps to be performed in the plurality of chambers 14-21 to be processed by the sample distribution unit 12, the processing steps comprising one or more processing pipetting steps by the sample distribution unit 12 and one or more processing incubation steps of the reaction mixture within any of the chambers 14-21.

The method is based on the inventive concept that the step of controlling 106 comprises setting or programming one of multiple, distinct test protocols 8-11. As such, the control unit 4 may select a test protocol 8-11 based on the specific assay process to be used.

FIG. 3 is an example of a cartridge 201 to be used with the point-of-care biomarker assay apparatus 1 according to the present invention.

The cartridge 201 comprises multiple chambers 203, 204, 205, 206, 208 (not all chambers have a reference sign) designed for receiving a plurality of liquid media. For example, the first chamber 203 is arranged for receiving the sample containing the biomarker. The second chamber 204 may be arranged for receiving the labelled binding reagent which is capable of binding to the biomarker. The third chamber 205 may contain magnetic beads reagent capable of binding to the biomarker and/or capable of binding competing binding reagent. The fourth chamber 206 may contain a wash buffer. A photo detector assembly may be arranged for measuring the presence or concentration of labelled binding reagent bound to the biomarker in the last chamber 208 of the cartridge 201.

The cartridge may further comprise a tag 207 in the form of a barcode or the like, which tag 207 may be read out by wireless sensor means comprised in the point-of-care biomarker assay apparatus. Based on the read out tag 207, the control unit may automatically set a specific test protocol. As such, the tag 207 may comprise information concerning what test protocol to be performed for the sample contained in the cartridge 201.

A pipet tip 202 is provided in the cartridge 201, which pipet tip 202 is arranged to be coupled to an hollow elongated tube present on the sample distribution unit. The sample distribution unit may autonomously and automatically couple the hollow elongated tube to the pipet tip 202 before the test protocol is initiated. After completion of the test protocol, the pipet tip 202 may be replaced, i.e. disposed, in the cartridge 201. The complete cartridge 201, including the pipet tip 202, may then be disposed of. The advantage hereof is that the point-of-care biomarker apparatus does not need to be equipped with pipet tips, as these pipet tips are readily available in the cartridge 201.

In the context of the present invention, a module, device, equipment, or the like may also be implemented as a computer program running on the processor.

The present invention is not limited to the embodiments as disclosed above, and can be modified and enhanced by those skilled in the art beyond the scope of the present invention as disclosed in the appended claims without having to apply inventive skills.

1-14. (canceled)

15. A point-of-care biomarker assay apparatus arranged for measuring a presence or concentration of a biomarker in a sample, comprising:
cartridge receiving means arranged for receiving at least one cartridge having chambers for receiving a plurality of liquid media comprising:
(i) the sample comprising the biomarker;
(ii) a labelled binding reagent capable of binding to the biomarker or capable of competing with the biomarker;
(iii) a magnetic beads reagent capable of binding to the biomarker and/or capable of binding a competing binding reagent;
(iv) a wash buffer; and
(v) at least one substrate capable of initiating a light emission from the labelled binding reagent;
a sample distribution unit arranged for processing pipetting steps with the chambers, thereby providing a liquid reactant mixture in one or more of the chambers, wherein the biomarker is allowed to bind to the labelled binding reagent and the magnetic beads reagent in a processing incubation step or to compete with the labelled binding reagent and bind the magnetic bead reagent in a processing incubation step;
wherein the sample distribution unit further comprises at least one precision pipet for performing the pipetting steps, each of the at least one precision pipet comprising:
(i) a hollow elongated tube arranged to detachably couple to a pipet tip provided at the at least one cartridge; and
(ii) an adjustable volume container, coupled to the hollow elongated tube, wherein the sample distribution unit is arranged to set a volume of the adjustable volume container for pipetting a set volume of liquid media from the chambers;
wherein the sample distribution unit is arranged to dispose the pipet tip at the at least one cartridge after the pipetting steps have been performed;
a magnetic coil assembly arranged for applying a magnetic field to the liquid reactant mixture for separating biomarkers bound to the magnetic beads reagent and/or the labelled binding reagent, from the reactant mixture;
a photo multiplier tube assembly arranged for measuring the presence or concentration of the labelled binding reagent bound to the biomarker, or bound to the magnetic beads in case of a competitive assay format;
a control unit arranged for controlling the processing pipetting steps with the chambers, and for controlling the sample distribution unit along the chambers according to a test protocol, wherein the test protocol comprises an order of subsequent processing steps performed in the plurality of chambers to be processed by the sample distribution unit, the processing steps comprising one or more processing pipetting steps by the sample distribution unit and one or more processing incubation steps of the reaction mixture within any of the chambers;
wherein the control unit is programmed for performing multiple, distinct test protocols; and
heating means arranged for heating each of the chambers of the at least one cartridge, simultaneously and uniformly, when placed in the apparatus, to a predetermined temperature.

16. The point-of-care biomarker assay apparatus according to claim 15, wherein the heating means comprises a plurality of metal heating elements arranged to be in contact with the at least one cartridge, when received in the cartridge receiving means, wherein the heating means are arranged to heat a received cartridge to the predetermined temperature by heating the metal heating elements.

17. The point-of-care biomarker assay apparatus according to claim 15, wherein the predetermined temperature is between 28 and 40 degrees Celsius.

18. The point-of-care biomarker assay apparatus according to claim 15, wherein the predetermined temperature is between 34 and 38 degrees Celsius.

19. The point-of-care biomarker assay apparatus according to claim 15, wherein the predetermined temperature is between 30 and 95 degrees Celsius.

20. The point-of-care biomarker assay apparatus according to claim 15, wherein the predetermined temperature is between 45 and 65 degrees Celsius.

21. The point-of-care biomarker assay apparatus according to claim 15, wherein the adjustable volume container comprises an adjustable volume between 5 μl and 1000 μl.

22. The point-of-care biomarker assay apparatus according to claim 15, wherein the sample distribution unit is arranged to set a volume of the adjustable volume container, for each pipetting step separately, for pipetting a set volume of liquid media from the chambers.

23. The point-of-care biomarker assay apparatus according to claim 15, wherein the assay apparatus comprises the at least one cartridge, and wherein the pipet tips provided at the at least one cartridge are arranged to provide level detection for detecting an amount of liquid media in the chambers and to provide clot detection for detecting clots in the liquid media.

24. The point-of-care biomarker assay apparatus according to claim 23, wherein the assay apparatus is arranged to provide an alert signal when a detected amount of liquid media in any of the chambers is below a predetermined threshold.

25. The point-of-care biomarker assay apparatus according to claim 23, wherein the assay apparatus is arranged to provide a clot alert signal when detected clots in the liquid media in any of the chambers are above a predetermined threshold.

26. The point-of-care biomarker assay apparatus according to claim 15, wherein the control unit is programmed to set a duration of the at least one washing step.

27. The point-of-care biomarker assay apparatus according to claim 15, further comprising a wireless sensor for wirelessly reading a tag provided on the at least one cartridge.

28. The point-of-care biomarker assay apparatus according to claim 27, wherein the control unit is programmed to set a specific test protocol based on the read tag.

29. The point-of-care biomarker assay apparatus according to claim 27, wherein the wireless sensor is adapted to read an RFID tag on the at least one cartridge.

30. A method for measuring a presence or concentration of a biomarker in a sample using a point-of-care biomarker assay apparatus, the method comprising the steps of:
receiving, with a cartridge receiving means, at least one cartridge having chambers for receiving a plurality of liquid media comprising:
(i) the sample comprising the biomarker;
(ii) a labelled binding reagent capable of binding to the biomarker;
(iii) a magnetic beads reagent capable of binding to the biomarker;
(iv) a wash buffer; and
(v) at least one substrate capable of initiating a light emission from the labelled binding reagent;
processing, with a sample distribution unit, pipetting steps with the chambers, thereby providing a liquid reactant mixture in one or more of the chambers, wherein the biomarker is allowed to bind to the labelled binding reagent and the magnetic beads reagent in a processing incubation step;
applying, with a magnetic coil assembly, a magnetic field to the liquid reactant mixture for separating biomarkers bound to magnetic beads and the labelled binding reagent, from the reactant mixture;
measuring, with a multiplier tube assembly, the presence or concentration of the labelled binding reagent bound to the biomarker;
controlling, with a control unit, the processing pipetting steps with the chambers, and for controlling the sample distribution unit along the chambers according to a test protocol comprising an order of subsequent processing steps performed in the chambers to be processed by the sample distribution unit, the processing steps comprising one or more processing pipetting steps by the sample distribution unit and one or more processing incubation steps of the reaction mixture within any of the chambers; and
heating the received at least one cartridge to a predetermined temperature;
wherein the control unit is programmed to perform multiple, distinct test protocols.
